(19) United States
(12) Patent Application Publication
(10) Pub. No.: US 2009/0036929 A1
(43) Pub. Date: Feb. 5, 2009

(54) Offset connector for a spinal stabilization rod

(76) Inventors: Joey Camia Reglos, Lake Forest, CA (US); Stanley Kyle Hayes, Mission Viejo, CA (US); Moti Altarac, Irvine, CA (US)

Correspondence Address: RIMAS LUKAS;VERTIFLEX, INC., 1351 CALLE AVANZADO, SAN CLEMENTE, CA, 92673, US

(21) Appl. No.: 12/249,203

(22) Filed: Oct. 10, 2008

Related U.S. Application Data
(63) Continuation-in-part of application No. 11/801,186, filed on May 9, 2007.
Application No. 11/801,186 is a continuation-in-part of application No. 11/801,194, filed on May 9, 2007.
Application No. 11/801,194 is a continuation-in-part of application No. 11/801,319, filed on May 9, 2007.
Application No. 11/801,319 is a continuation-in-part of application No. 11/726,093, filed on Mar. 20, 2007.
Application No. 11/726,093 is a continuation-in-part of application No. 11/586,849, filed on Oct. 25, 2006.
Application No. 11/586,849 is a continuation-in-part of application No. 11/362,366, filed on Feb. 23, 2006.
Application No. 11/362,366 is a continuation-in-part of application No. 11/427,738, filed on Jun. 29, 2006.
Application No. 11/427,738 is a continuation-in-part of application No. 11/436,407, filed on May 17, 2006.
(60) Provisional application No. 60/998,620, filed on Oct. 12, 2007.
(60) Provisional application No. 60/701,660, filed on Jul. 22, 2005.

(51) Int. Cl. A61B 17/70; A61B 17/04

(52) U.S. Cl. 606/278; 606/264; 606/301

(57) ABSTRACT

An offset connector implantable into a patient and connectable between a vertebral anchor a spinal stabilization rod is provided. The offset connector for connecting a spinal stabilization rod to a bone anchor system is laterally displaced relative to the rod and movable with respect to the bone anchor system until positioned and locked in place. The offset connector includes a stem configured for attachment to the bone anchor system and a rod-receiving portion connected to the stem. The rod-receiving portion is configured to receive at least a portion of the spinal stabilization rod at a location displaced from the bone anchor system. A fastener mechanism configured to secure the spinal stabilization rod to the rod-receiving portion is also provided.

### CROSS-REFERENCE TO RELATED APPLICATIONS

This application claims priority to and the benefit of and is a continuation-in-part of U.S. Provisional Patent Application Ser. No. 60/998,620 entitled “Offset connector for a spinal stabilization rod” filed on Oct. 12, 2007 which is hereby incorporated herein by reference in its entirety. This patent application is also a continuation-in-part of U.S. patent application Ser. No. 11/801,186 entitled “Systems and methods for posterior dynamic stabilization of the spine” filed on May 9, 2007 and a continuation-in-part of U.S. patent application Ser. No. 11/801,194 entitled “Systems and methods for posterior dynamic stabilization of the spine” filed on May 9, 2007 and a continuation-in-part of U.S. patent application Ser. No. 11/801,319 entitled “Systems and methods for posterior dynamic stabilization of the spine” filed on May 9, 2007 all of which are incorporated herein by reference in their entireties. This application is also a continuation-in-part of U.S. patent application Ser. No. 11/726,093 entitled “Screw systems and methods for use in stabilization of bone structures” filed on Mar. 20, 2007, and a continuation-in-part of U.S. patent application Ser. No. 11/586,849 entitled “Systems and methods for stabilization of bone structures” filed on Oct. 25, 2006, and a continuation-in-part of U.S. patent application Ser. No. 11/362,366 entitled “Systems and methods for stabilization of bone structures” filed on Feb. 23, 2006, which is a continuation-in-part of U.S. Provisional Patent Application No. 60/701,660 entitled “Systems and methods for stabilization of bone structures” filed on Jul. 22, 2005 all of which are incorporated herein by reference in their entireties. This application is also a continuation-in-part of U.S. patent application Ser. No. 11/427,738 entitled “Systems and methods for stabilization of bone structures” filed on Jun. 29, 2006, and a continuation-in-part of U.S. patent application Ser. No. 11/436,407 entitled “Systems and methods for posterior dynamic stabilization of the spine” filed on May 17, 2006, all of which are hereby incorporated herein by reference in their entireties.

### FIELD

The present invention generally relates to devices, systems, and methods for the fixation of the spine. In particular, the present invention relates to an offset connector for connecting a spinal fixation member such as a rod to a bone anchor such as a screw.

### BACKGROUND

The spinal column is a highly complex system of bones and connective tissues that provides support for the body and protects the delicate spinal column and nerves. The spinal column includes a series of vertebrae stacked one atop the other, whereby each vertebral body includes a relatively strong bone portion (cortical) forming the outside surface of the body and a relatively weak bone portion (cancellous) forming the center of the body. Situated between each vertebral body is an intervertebral disc formed from a non-bony, fibro-cartilage material that provides for cushioning and dampening of compressive forces applied to the spinal column. The vertebral canal containing the delicate spinal cords and nerves is located just posterior to the vertebral bodies.

Various types of spinal column disorders are known and include scoliosis (abnormal lateral curvature of the spine), kyphosis (abnormal forward curvature of the spine, usually in the thoracic spine), excess lordosis (abnormal backward curvature of the spine, usually in the lumbar spine), spondylolisthesis (forward displacement of one vertebra over another, usually in a lumbar or cervical spine) and other disorders caused by abnormalities, disease or trauma, such as ruptured or slipped discs, degenerative disc disease, fractured vertebra, and the like. Patients suffering from such conditions usually experience extreme and debilitating pain as well as diminished nerve function.

One technique for remedying such conditions is spinal fixation. In spinal fixation surgical implants are used for fusing together and/or mechanically immobilizing adjacent vertebrae of the spine. Once the spinal fixation system has been assembled and fixed to a series of two or more vertebrae, it constitutes a rigid device preventing the vertebrae from moving relative to one another. This rigidity enables the implanted system to support all or part of the stresses instead of the stresses being born by the series of damaged vertebrae.

Spinal fixation may also be used to alter the alignment of the adjacent vertebrae relative to one another so as to alter the overall alignment of the spine. Such techniques have been used effectively to treat the above-described conditions and, in most cases, to relieve pain suffered by the patient. One particular spinal fixation method involves orthopedic rods affixed generally parallel to the spine. This is accomplished by fastening bone screws to the posteriorly projecting pedicles of the appropriate vertebrae. Bone screws may be delivered in a percutaneous, minimally-invasive or open procedure. The pedicle screws are generally placed two per vertebra, one at each pedicle on either side of the spinous process, and serve as anchor points for the spinal rods. The distance between pedicles of the same vertebral body slightly increases with each vertebra down the spinal column in the lumbar region. As a result of this increased distance, a rod affixed directly to a bone screw will be angled slightly outwardly and in some cases, especially across more than one level, a rod will be located laterally inwardly of the pedicles to preserve a substantial vertical and non-angled orientation of the rod and reduce stress concentrations arising from an overly-angled rod. A connector bridges this lateral displacement to connect the rod to the pedicle bone anchor. Connectors are typically adapted for receiving a spinal rod at one end and connecting to the pedicle screw at the other end. When implanted, the aligning influence of the rods forces the spine to conform to a more desirable shape. In certain instances, the spine rods may be bent to achieve a desired localized curvature of the spinal column.

This invention relates generally to improvements in spinal fixation devices of the type designed particularly for human implantation, to maintain the adjacent spinal vertebrae in a substantially fixed and predetermined spatial relation while, if desired, promoting bone ingrowth and fusion therebetween. More particularly, this invention relates to an improved system including more than one poly-axial pedicle screw units in combination with an elongated and interconnecting stabilizer rod which is offset relative to at least one pedicle screw unit. The offset rod is connected to the at least one screw unit via an offset connector which joins the rod to the screw unit.

### SUMMARY

According to one aspect of the invention an offset connector for connecting a spinal stabilization rod to a bone anchor system that is substantially laterally displaced relative to the rod is disclosed. The offset connector includes a stem configured for attachment to the bone anchor system. A rod-receiving portion is connected to the stem and configured to receive at least a portion of the spinal stabilization rod at a location displaced from the bone anchor system. The offset connector further includes a fastener mechanism configured to secure the spinal stabilization rod to the rod-receiving portion. The stem is movable with respect to the bone anchor system to change the distance between the rod-receiving portion and the bone anchor system.

According to another aspect of the invention, a spinal fixation system for the stabilization of two or more vertebral bodies is disclosed. The system includes at least two bone anchors. One bone anchor is implantable in one vertebral body and the other bone anchor is implantable in another vertebral body. Each of the bone anchors include a first receiving portion connected to a threaded shank portion. The system includes at least one elongated member for interconnecting the vertebral bodies. The system includes a connector having a second receiving portion connected to a stem. The second receiving portion is configured to receive a portion of the elongated member and connect thereto. The first receiving portion is configured to receive either a portion of the stem or a portion of the elongated member and connect thereto. The first receiving portion of one bone anchor is connected to the stem and the first receiving portion of the other bone anchor is connected to the elongated member.

According to another aspect of the invention, a method for implanting a spinal implant system in a patient's spine is disclosed. A first bone anchor having a first shank connected to a first channel that is closable with a first fastener mechanism is provided. The first bone anchor is implanted in a first vertebral body of the spine along one side of the spinous process. A second bone anchor having a second shank connected to a second channel that is closable with a second fastener mechanism is provided. The second bone anchor is implanted in a second vertebral body of the spine along the same side of the spine as the first bone anchor. A cross connector having a third channel is provided. The cross connector has a stem connected to the third channel. The stem is inserted into the second channel. An elongated member is provided. A portion of the elongated member is inserted into the first channel. The stem is moved inside the second channel to position the elongated member inside the third channel. A portion of the elongated member is inserted into the third channel.

Other advantages will be apparent from the description that follows, including the drawings and claims.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 illustrates a posterior view of a portion of a patient's spine implanted with bone anchors, rods and offset connectors.

FIG. 2a illustrates a perspective and partially exploded view of a bone anchor and offset connector system according to the present invention.

FIG. 2b illustrates a side and partially exploded view of a bone anchor and offset connector system according to the present invention.

FIG. 2c illustrates a top view of a bone anchor and offset connector system according to the present invention.

FIG. 3a illustrates a perspective view of a bone anchor and offset connector system connected to a rod according to the present invention.

FIG. 3b illustrates a side view of a bone anchor and offset connector system connected to a rod according to the present invention.

FIG. 3c illustrates a perspective view of an offset connector according to the present invention.

FIG. 4a illustrates a perspective view of a bone anchor and offset connector system connected to a rod according to the present invention.

FIG. 4b illustrates a side view of a bone anchor and offset connector system connected to a rod according to the present invention.

FIG. 4c illustrates a perspective view of an offset connector according to the present invention.

FIG. 5a illustrates a perspective view of a bone anchor and offset connector without a closure mechanism according to the present invention.

FIG. 5b illustrates a side and exploded view of a bone anchor and offset connector system according to the present invention.

FIG. 5c illustrates a side crossectional view of a bone anchor and offset connector without a closure mechanism according to the present invention.

### DETAILED DESCRIPTION

Before the subject devices, systems and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a spinal segment” may include a plurality of such spinal segments and reference to “the screw” includes reference to one or more screws and equivalents thereof known to those skilled in the art, and so forth.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The present invention is described in the accompanying figures and text as understood by a person having ordinary skill in the field of spinal implants.

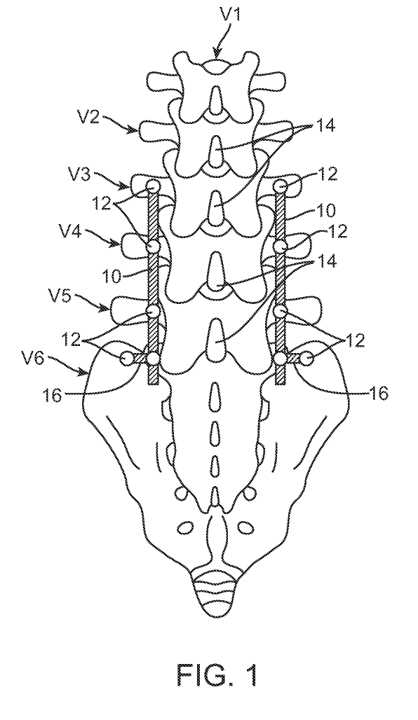

Referring now to FIG. 1, there is shown a portion of a spinal column with vertebral bodies V1-V6. Vertebral bodies V3 through V6 are fixed with two spinal rods or elongated members 10 connected to bone screws 12 implanted in the pedicles of the vertebrae on both sides of the spinous processes 14. An offset connector 16 is employed to connect the elongated member 10 to the outwardly (laterally) displaced bone screws 12 of vertebral body V6. The invention is not limited to the specific vertebral placement shown in FIG. 1. FIG. 1 is employed for illustrative purposes showing one example where an offset connector may be used. An orthopedic spinal neurosurgeon having ordinary skill in the art would understand the range of possible use and placement of the offset connector of the present invention.

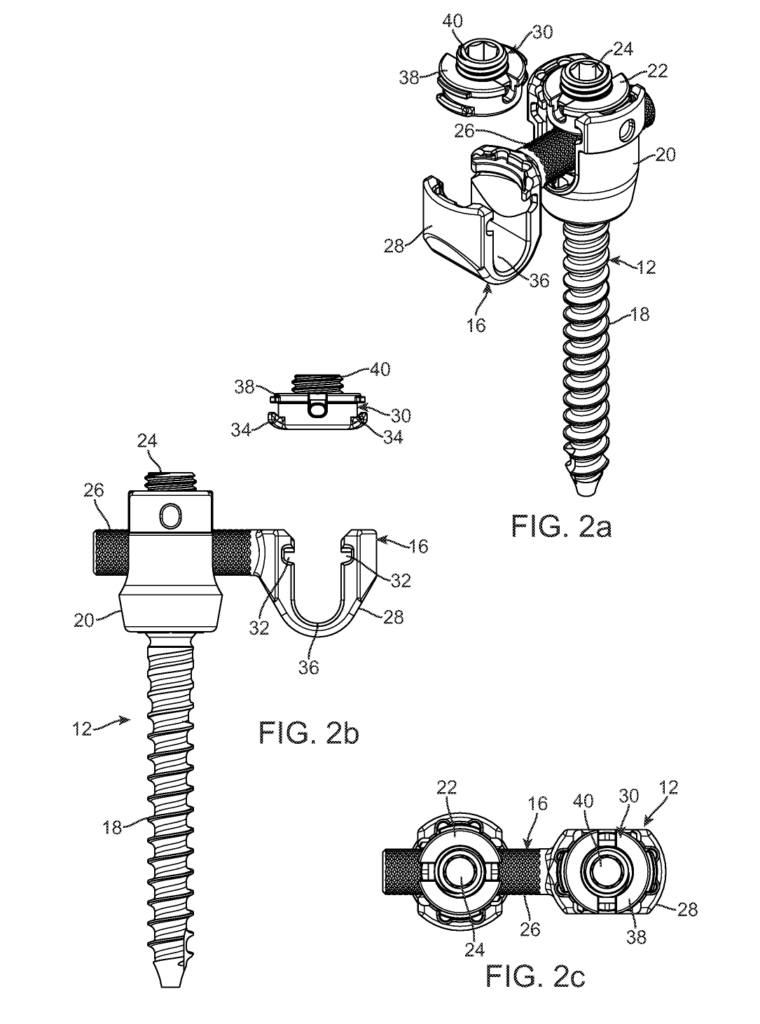

Referring now to FIGS. 2a, 2b and 2c, there is shown an offset connector 16 according to the present invention. The offset connector 16 is connected to a bone anchor system 12. A spinal rod is not shown. A spinal fixation system generally includes a first set of two bone anchor systems installed into the pedicles of a superior vertebral segment, a second set of two bone anchor systems installed into the pedicles of an inferior vertebral segment, a first link element connected between one of the pedicle bone anchor systems in the first set and one of the pedicle bone anchor systems in the second set along the same side of the inferior and superior vertebral segments, and a second link element connected between the other of the pedicle bone anchor systems in the first set and the other of the pedicle bone anchor systems in the second set along the same side of the inferior and superior vertebral segments. At least a pair of vertebral bodies is thereby linked and fixed together to maintain the skeletal structures in a spaced relation while promoting bone ingrowth and fusion between vertebral bodies. In general, the fixation system provides a strong mechanical load bearing structure with at least one rod 10 connecting with two or more bone screw units 12 secured or anchored to the vertebrae.

A typical anchor system 12 comprises, but is not limited to, a spinal bone screw 18 that is designed to have one end or shank that inserts threadably into a vertebra and another end that is generally spherical in shape for polyaxial mating with a seat 20 connected at the opposite end thereof. Typically, the seat 20 is designed to receive the link element in a channel in the seat. The link element is typically a rod or rod-like member or elongated member. The seat typically has two upstanding arms that are on opposite sides of the channel that receives the rod member. The rod is laid in the open channel which is then closed with a closure member, fastener mechanism, to both capture the rod in the channel and lock it in the seat to prevent relative movement between the seat and the rod. In cases where the placement of the screws 12 as dictated by patient anatomy result in a lateral displacement from a rod (as shown in FIG. 1 for example), an offset connector 16, instead of a rod 10, is located in the seat 20 of the anchor system 12 and secured therein with a cap 22 and set screw 24.

Still referencing FIGS. 2a-2c, the offset connector 16 includes a stem 26 connected to a rod receiving portion 28 or otherwise called an elongated member-receiving portion 28 and a closure/locking mechanism 30 or otherwise called a fastener mechanism 30 which is configured to close the rod-receiving portion 28 and secure the rod 10 therein. The stem 26 is an extension that is substantially rod-like in shape and configured to fit inside the channel of the seat 20 of the anchor system 12. The displacement of the offset connector 16 relative to the anchor system 12 is adjustable by moving the stem 26 inside the channel of the seat 20 and locking it in the desired position with the cap 22 and set screw 24. The rod-receiving portion 28 of the offset connector 16 includes an open channel 36 that in one variation is substantially U-shaped. The channel 36 is configured to receive at least a portion of the rod 10 therein. The rod-receiving portion 28 includes grooves 32 for receiving locking lugs 34 of the fastener mechanism 30. The locking lugs 34 are angled upwardly as seen in FIG. 2b to advantageously prevent outward splaying of the U-shaped channel 36. The closure mechanism 30 includes a cap 38 and set screw 40. In use, a rod 10 (not shown) is positioned inside the rod-receiving portion 28 of the offset connector 16 and the closure or fastener mechanism 30 is oriented such that the locking lugs 34 are aligned with the open U-shaped channel 36. The cap 38 is inserted into the upwardly or posteriorly facing U-shaped channel 36 and rotated by approximately 90 degrees to secure the cap 38 to the rod-receiving portion 28. In one variation, with the cap 38 in place, the rod is permitted to slide within the rod-receiving portion 28. A set screw 40 is advanced through the cap 38 to bear down on the rod 10 and lock/secure it in place. In the variation in which the rod is permitted to slide within the rod-receiving portion 28 with the cap 38 in place, the set screw 40 arrests such relative motion when advanced onto the rod 10.

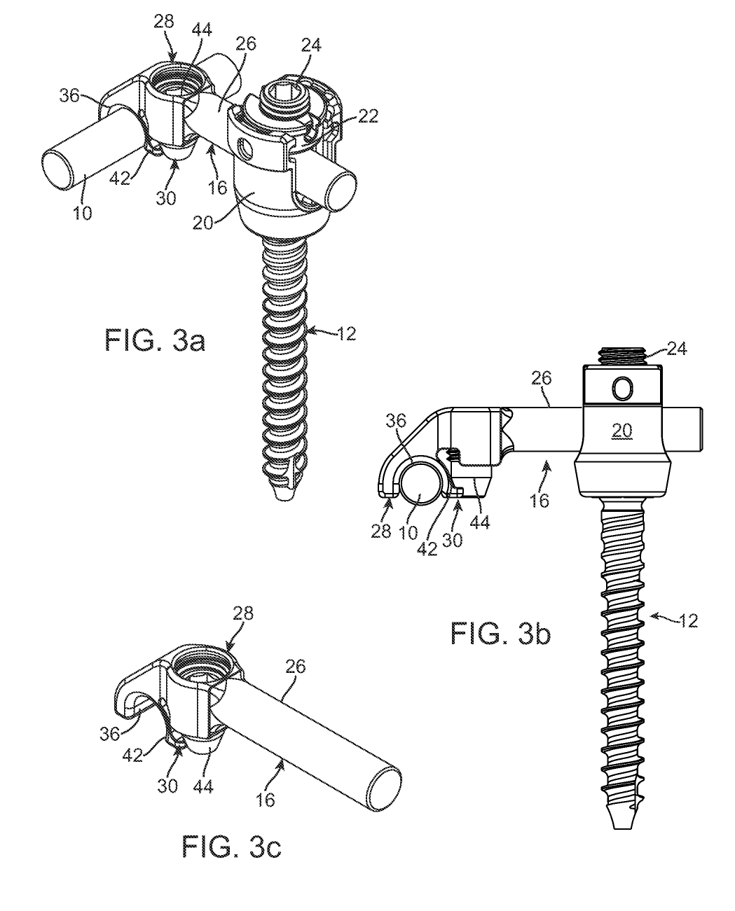

Turning now to FIGS. 3a, 3b and 3c, there is shown a top-loading offset connector 16 according to the present invention shown connected to a bone anchor system 12 (FIGS. 3a and 3b) as described above wherein like reference numerals are used to describe like parts. The offset connector 16 includes a stem 26 connected to a rod-receiving portion 28 and a closure mechanism 30, or otherwise called a locking or fastener mechanism 30. The stem 26 is an extension that is substantially rod-like in shape and configured to fit inside the channel of the seat 20 of the anchor system 12. The displacement of the offset connector 16 relative to the anchor system 12 is adjustable by moving the stem 26 inside the channel of the seat 20 and locking it in the desired position with the cap 22 and set screw 24. The rod-receiving portion 28 of the offset connector 16 includes an upside-down or downwardly or anteriorly open channel 36 that is substantially U-shaped in one variation. The channel 36 is configured to receive at least a portion of the rod 10 therein. The rod-receiving portion 28 includes a bias portion 42. The fastener mechanism 30 includes a locking screw 44 having a threaded portion and a bearing portion. When advanced inside the fastener mechanism 30, the bearing portion of the locking screw 44 deflects the bias portion 42 which contacts the rod 10 locking it in place, arresting movement of the rod 10 relative to the offset connector 16.

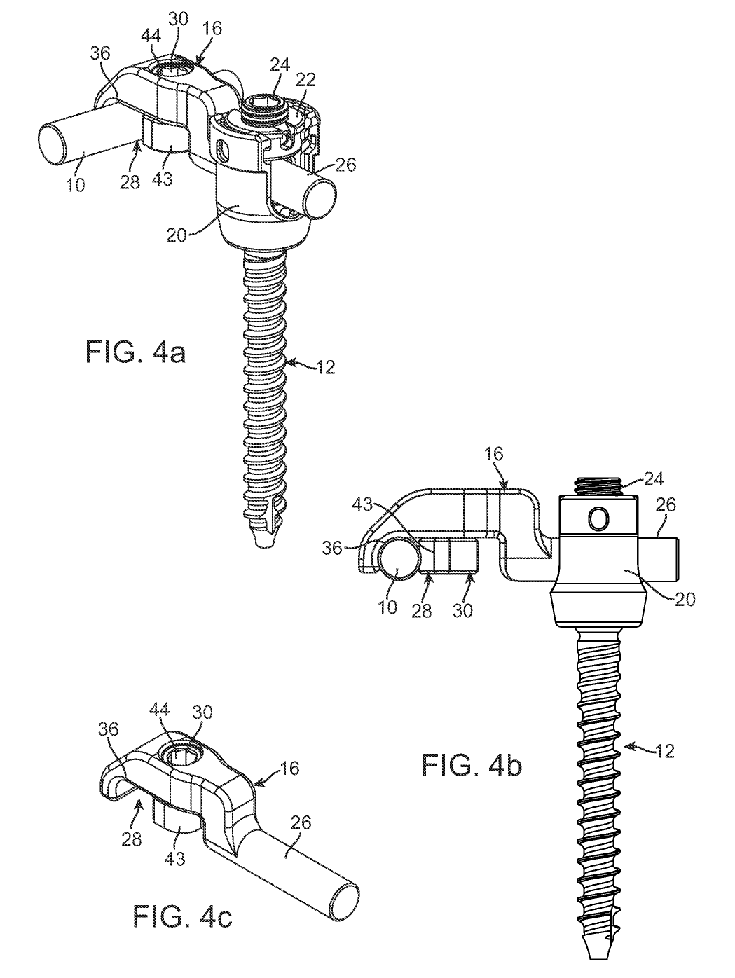

Turning now to FIGS. 4a, 4b and 4c, there is shown another top-loading offset connector 16 according to the present invention shown connected to a bone anchor system 12 (FIGS. 4a and 4b) as described above wherein like reference numerals are used to describe like parts. The offset connector 16 includes a stem 26 connected to a rod-receiving portion 28 and a closure/locking/fastener mechanism 30. The stem 26 is an extension that is substantially rod-like in shape and configured to fit inside the channel of the seat 20 of the anchor system 12. The displacement of the offset connector 16 relative to the anchor system 12 is adjustable by moving the stem 26 inside the channel of the seat 20 and locking it in the desired position with the cap 22 and set screw 24. The rod-receiving portion 28 of the offset connector 16 includes a substantially upside-down or downwardly or anteriorly facing U-shaped channel 36 configured to receive at least a portion of the rod 10 therein. With particular reference to FIG. 4b, the offset connector 16 is configured such that the rod 10 and stem 26 are substantially coplanar, unlike the variations shown in FIGS. 2a-3c wherein the rod 10 lies substantially in a plane beneath the plane of the stem 26. A portion of the U-shaped channel is formed by the lower end (caming portion 43) of the locking mechanism 30. The fastener mechanism 30 includes a locking screw 44 (shown in FIGS. 4a and 4c) having a threaded portion and a caming portion 43. When advanced inside the fastener mechanism 30, the caming portion 43 rotates and bears against the rod 10 locking it in place, arresting movement of the rod 10 relative to the offset connector 16.

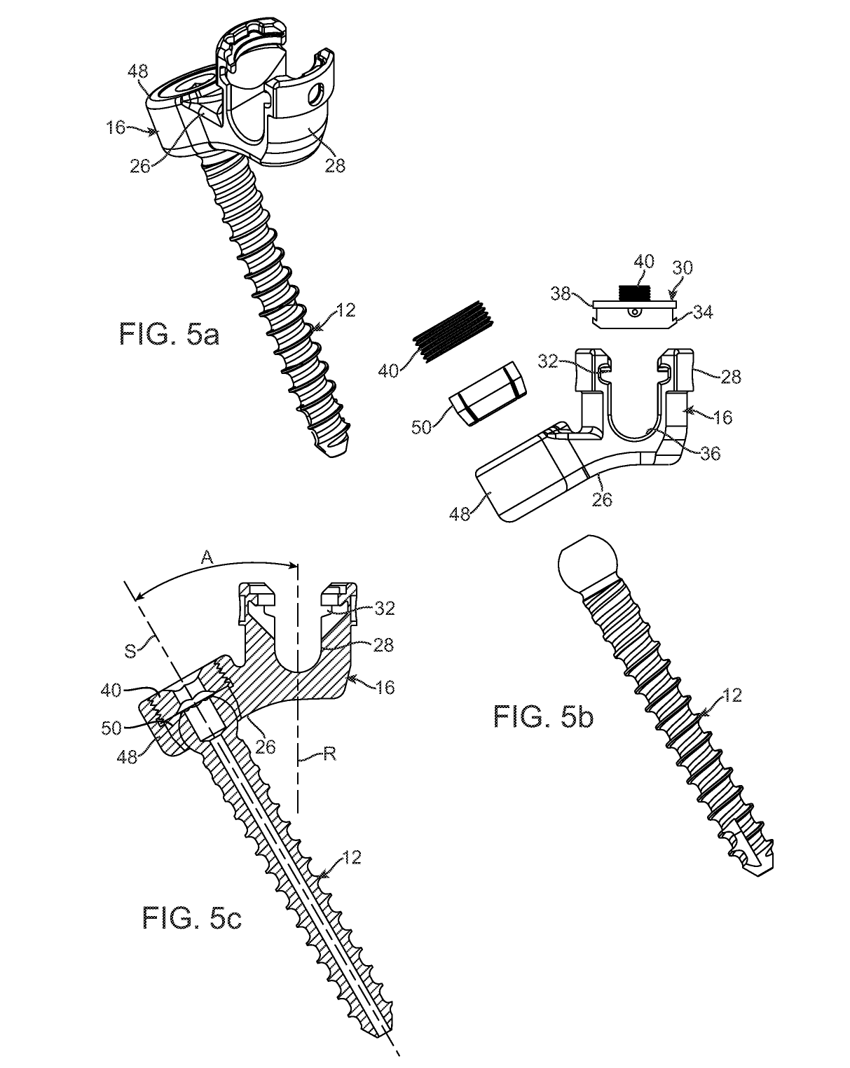

Turning now to FIGS. 5a, 5b and 5c there is an offset connector 16 connected to a bone anchor 12. The offset connector 16 includes a stem 26 connected to a rod-receiving portion 28 and a closure/locking/fastener mechanism 30 (shown in FIG. 5b) which is configured to close the rod-receiving portion 28 and secure the rod 10 (not shown) therein. The rod-receiving portion 28 of the offset connector 16 includes a substantially U-shaped channel 36 configured to receive at least a portion of the rod 10 therein. The U-shaped channel has a longitudinal axis R shown as a dashed line in FIG. 5c. The rod-receiving portion 28 includes grooves 32 for receiving locking lugs 34 of the closure/locking/fastener mechanism 30. The locking lugs 34 are angled upwardly as seen in FIG. 5b to advantageously prevent splaying of the U-shaped channel 36. The fastener mechanism 30 includes a cap 38 and set screw 40. The stem 26 includes a bone screw-receiving portion 48 that comprises a bore having a longitudinal axis S shown as a dashed line in FIG. 5c. The offset connector 16 is angled such that the longitudinal axis R of the rod-receiving portion 28 is angled with respected to the longitudinal axis S of the bone screw-receiving portion 48 by an angle A. Angle A is approximately between 10 degrees and 80 degrees. The bore of the bone screw receiving portion 48 is configured to capture the substantially spherical ball head of the bone screw. A retainer 50 and set screw 40 inserted into the bore locks the polyaxial bone screw in place. In use, the bone screw 12 is passed through the bore of the bone screw-receiving portion 48 and the retainer 50 is disposed inside the bore between the head of the screw and the bone-screw receiving portion 48. The set screw 40 is threaded inside the bore but not tightened all the way to lock the bone screw in position. Thereby, the bone screw 12 is permitted to angulate polyaxially with respect to the connector 16. When the desired angle is established the set screw 40 is advanced and tightened to lock the bone screw 12 in position. A rod 10 (not shown) is positioned inside the rod-receiving portion 28 of the offset connector 16 and the fastener mechanism 30 is oriented such that the locking lugs 34 are aligned with the open U-shaped channel 36. The cap 38 is inserted into the U-shaped channel 36 and rotated by approximately 90 degrees to secure the cap 38 to the rod-receiving portion 28. A set screw 40 is advanced through the cap 38 to bear down on the rod 10 and secure it in place, arresting movement of the rod 10 relative to the offset connector 16.

As seen in the variations illustrated in FIGS. 2, 3 and 4, the bone anchor 12 and offset connector 16 system are configured such that the same bone anchor 12 can be utilized for implantation in any of the vertebrae of the spine such as V1-V6 of FIG. 1, including the offset vertebral location V6. Hence, the system advantageously does not require different bone anchors 12, ones for offset vertebral locations and different ones for non-offset vertebral locations, to be delivered to the surgeon. The surgeon may easily implant all of the bone anchors 12 in the appropriate locations. The system advantageously and easily allows the surgeon to employ the offset connector 16 with any of the same bone anchors 12 wherever an offset location arises. Furthermore, the offset connector 16 is movable with respect to the bone anchor 12 to adjust the degree of offset as needed by sliding the connector 16 back or forth within the bone anchor 12. In essence, the seat of the bone anchor is configured to receive either the rod 10 or the stem 26. In other words, the bone anchor and connector system of the present invention is configured with two seats wherein one seat on the bone anchor is configured to receive either a rod or the other seat in a displaced relationship in which the other seat is configured to receive the rod. This configuration of a channel within a channel is particularly advantageous and affords greater flexibility for the surgeon during the operation.

In use, a first bone anchor having a first threaded shank connected to a first channel that is closable with a first fastener mechanism is provided. Of course, the first channel may be polyaxially connected to the first shank. The first bone anchor is implanted in a first vertebral body of the spine along one side of the spinous process. A second bone anchor having a second threaded shank connected to a second channel that is closable with a second fastener mechanism is provided. Of course, the second channel may be polyaxially connected to the second shank. The second bone anchor is implanted in a second vertebral body of the spine along the same side of the spine as the first bone anchor. A cross connector having a third channel is provided. The cross connector has a stem connected to the third channel and in one variation, polyaxially connected thereto. The stem is inserted into the second channel. An elongated member or rod is provided. A portion of the elongated member is inserted into the first channel. The stem is moved inside the second channel, back and forth as necessary, to position the elongated member inside the third channel. If necessary, the elongated member is moved within the first channel, back and forth as necessary to place the elongated member in the third channel wherein it may also be moved back and forth as necessary to position and place the rod and interconnect the first and second vertebral bodies with the elongated member via the connector. A portion of the elongated member is inserted into the third channel. Fastener mechanisms are employed to retain and/or lock the elongated member in the appropriate channels. For example, a first fastener mechanism is used to retain the elongated member in the first channel. In one variation, the first fastener mechanism retains the elongated member in the first channel such that the elongated member is permitted to slide within the channel for positioning. When in position, in one variation, the first fastener mechanism is operable to arrest the relative motion of the elongated member with respect to the first channel. And in another variation, the first fastener mechanism is operable to arrest polyaxial motion of the first channel relative to the shank. Similarly, a second fastener mechanism may be employed to capture the stem in the second channel in such a manner that the stem is capable of sliding with respect to the second channel. The movement of the stem relative to the second channel advantageously allows the distance of the third channel from the second channel to be changed to easily connect the entire construct together in anatomies that do not allow the first and second bone anchors to be perfectly in alignment for elongated member to easily attach directly to both without the employment of a cross-connector. When the stem is in position with respect to the second channel, the fastener mechanism is further operable to arrest the relative movement of the stem with respect to the second channel and in another variation, operable to arrest any polyaxial motion of the second channel relative to the second shank. With respect to the third channel and the elongated member positioned therein, a third fastener mechanism is employed to capture and retain the elongated member inside the third channel while permitting movement of the elongated member with respect to the third channel. When the elongated member is positioned with respect to the third channel the third fastener mechanism is operable to stop the movement of the elongated member with respect to the third channel. In one variation, the third fastener mechanism operates to also arrest any polyaxial motion of the third channel relative to the stem. With all the fastener mechanism employed, the entire construct is locked in place to stabilize the interconnected spinal vertebrae. The system advantageously employees bone anchors with channels configured to receive either an elongated member or a stem, therefore, different bone anchors need not be employed and the surgeon can determine which of the bone anchors will receive the stem of the connector as appropriate. Other advantages will be apparent from the description that follows, including the drawings and claims.

The disclosed devices or any of their components can be made of any biologically adaptable or compatible materials including PEEK, PEK, PAEK, PEKEKK or other polyetherketones. Materials considered acceptable for biological implantation are well known and include, but are not limited to, stainless steel, titanium, tantalum, combination metallic alloys, various plastics, polymers, resins, ceramics, biologically absorbable materials and the like. Any components may be also coated/made with osteo-conductive (such as deminerized bone matrix, hydroxyapatite, and the like) and/or osteo-inductive (such as Transforming Growth Factor “TGF-B,” Platelet-Derived Growth Factor “PDGF,” Bone-Morphogenic Protein “BMP,” and the like) bio-active materials that promote bone formation and oseo-integration. Further, a surface of any of the implants may be made with a porous ingrowth surface (such as titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone ingrowth or establish a mineralized connection between the bone and the implant, and reduce the likelihood of implant loosening. Lastly, any assembly or its components can also be entirely or partially made of a shape memory material or other deformable material.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

We claim:

1. An offset connector for connecting a spinal stabilization rod to a bone anchor system that is substantially laterally displaced relative to the rod comprising:
a stem configured for attachment to the bone anchor system;
a rod-receiving portion connected to the stem; the rod-receiving portion configured to receive at least a portion of the spinal stabilization rod at a location displaced from the bone anchor system; and
a fastener mechanism configured to secure the spinal stabilization rod to the rod-receiving portion;
wherein the stem is movable with respect to the bone anchor system to change the distance between the rod-receiving portion and the bone anchor system.

2. The offset connector of claim 1 wherein the bone anchor system further includes a closable channel configured for placement and securement of at least a portion of the stem within the channel.

3. The offset connector of claim 2 wherein the rod-receiving portion is configured to receive at least a portion of the spinal stabilization rod such that the at least a portion of the spinal stabilization rod that is received in the rod-receiving portion is substantially co-planar with that portion of the stem placed and secured within the channel of the bone anchor system.

4. The offset connector of claim 1 wherein the rod-receiving portion is a channel.

5. The offset connector of claim 4 wherein the channel opens upwardly or generally posteriorly.

6. The offset connector of claim 4 wherein the channel opens downwardly or generally anteriorly.

7. The offset connector of claim 1 wherein at least a portion of the rod-receiving portion includes a deflectable bias portion; the offset connector further including a fastener mechanism operable to deflect the deflectable bias portion to lock and secure the spinal stabilization rod to the rod-receiving portion.

8. The offset connector of claim 7 wherein the fastener mechanism comprises a locking screw having a threaded portion and a bearing portion; the bearing portion configured to deflect the bias portion to lock and secure the spinal stabilization rod to the rod-receiving portion when the locking screw is advanced in the connector.

9. The offset connector of claim 1 wherein the fastener mechanism comprises a locking screw having a threaded portion and a caming portion; the caming portion is configured to lock and secure the spinal stabilization rod to the rod-receiving portion when the locking screw is advanced in the connector to turn the caming portion to lock the spinal stabilization rod in place.

10. The offset connector of claim 1 wherein the bone anchor system further includes a fastener mechanism configured to lock movement of the stem relative to the bone anchor system.

11. A spinal fixation system for the stabilization of two or more vertebral bodies, comprising:
at least two bone anchors, one bone anchor implantable in one vertebral body and the other bone anchor implantable in another vertebral body; each bone anchor including a first receiving portion connected to a threaded shank portion;
at least one elongated member for interconnecting the vertebral bodies;
a connector including a second receiving portion connected to a stem; the second receiving portion is configured to receive a portion of the elongated member and connect thereto; and
the first receiving portion is configured to receive either a portion of the stem or a portion of the elongated member and connect thereto;
wherein the first receiving portion of one bone anchor is connected to the stem and the first receiving portion of the other bone anchor is connected to the elongated member.

12. The system of claim 11 wherein the first receiving portion of one bone anchor is connected to the stem and the first receiving portion of the other bone anchor is connected to the elongated member such that second receiving portion and the elongated rod connected thereto is displaced from the first receiving portion to which the stem is connected.

13. The system of claim 11 wherein the first receiving portion is polyaxially connected to the threaded shank portion.

14. The system of claim 11 wherein the second receiving portion is polyaxially connected to the stem.

15. The system of claim 11 wherein the first and second receiving portions are channels closable with fasteners to secure the elongated member or stem inside the channels.

16. The system of claim 11 wherein the first and second receiving portions are channels closable with fasteners configured firstly to retain the rod or stem inside the channel for adjusting their positioning and secondly to prevent relative motion of the rod or stem with respect to the channels.

17. A method for implanting a spinal implant system in a patient's spine comprising the steps of:
providing a first bone anchor having a first shank connected to a first channel closable with a first fastener mechanism;
implanting the first bone anchor in a first vertebral body of the spine along one side of the spinous process;
providing a second bone anchor having a second shank connected to a second channel closable with a second fastener mechanism;
implanting the second bone anchor in a second vertebral body of the spine along the same side of the spine as the first bone anchor;
providing a connector having a third channel; the connector having a stem connected to the third channel;
inserting the stem into the second channel;
providing an elongated member;
inserting a portion of the elongated member into the first channel;
moving the stem inside the second channel to position the elongated member inside the third channel;
inserting a portion of the elongated member into the third channel.

18. The method of claim 17 further including the step of closing the second channel with the second fastener mechanism with a portion of the stem located in the second channel such that the stem is movable within and with respect to the second channel.

19. The method of claim 17 further including the step of closing the first channel with the first fastener mechanism with a portion of the elongated member located in the first channel such that the elongated member is movable within and with respect to the first channel.

20. The method of claim 17 further including the step of closing the third channel with a third fastener mechanism with a portion of the elongated member located in the first channel such that the elongated member is movable with respect to the third channel.

21. The method of claim 17 further including the step of locking movement of the elongated member relative to the third channel.

22. The method of claim 21 wherein the step of locking movement of the elongated member relative to the third channel includes locking any polyaxial motion of the third channel relative to the stem.

23. The method of claim 17 further including the step of locking movement of the elongated member relative to the first channel with the first fastener mechanism.

24. The method of claim 23 wherein the step of locking movement of the elongated member relative to the first channel includes locking any polyaxial motion of the first channel relative to the first shank.

25. The method of claim 17 further including the step of locking movement of the stem relative to the second channel with the second fastener mechanism.

26. The method of claim 25 wherein the step of locking movement of the stem relative to the second channel includes locking any polyaxial motion of the second channel relative to the second shank.

27. The method of claim 17 further including the step of providing first and second bone anchors with first and second channels configured to receive the rod or the stem.
